(19) United States
(12) Patent Application Publication
(10) Pub. No.: US 2020/0230262 A1
(43) Pub. Date: Jul. 23, 2020

(54) COMPOSITION FOR IMPROVING ULTRASONIC TRANSMISSION EFFICIENCY, GEL COMPOSITION FOR ULTRASONIC DIAGNOSIS, AND ULTRASOUND IMAGING METHOD

(71) Applicant: HAKUTO CO., LTD., Tokyo (JP)

(72) Inventors: Yasuhiro NOHATA, Yokkaichi-shi, Mei (JP); Rie Ujihara, Yokkai-shi, Mei (JP); Ryoichi Saka, Tokyo (JP); Futoshi Ogata, Tokyo (JP)

(21) Appl. No.: 16/633,506

(22) PCT Filed: Jul. 6, 2018
(86) PCT No.: PCT/JP2018/025659

§ 371 Date: Jan. 23, 2020

(30) Foreign Application Priority Data
Jul. 25, 2017 (JP) 20107-143572

(51) Int. Cl. A61K 49/22; A61B 8/14

(52) U.S. Cl. CPC A61K 49/226; C08L 5/16; A61B 8/14

(57) ABSTRACT

An object is to provide an ultrasonic transmission efficiency improving composition for ultrasonic diagnosis which can suppress attenuation of ultrasonic waves in a skin stratum corneum to reach deeper portions of a living body, and has ultrasonic transmission efficiency stable even with temperature change during use; an ultrasonic diagnostic gel composition which is obtained by gelling the ultrasonic transmission efficiency improving composition into a gel state; and an imaging method using the ultrasonic transmission efficiency improving composition and the ultrasonic diagnostic gel composition.

The solution includes an ultrasonic transmission efficiency improving composition including an aqueous solution containing hydrophilic fine particles including glycogen and/or hydrophilic fine particles including dextrin; an ultrasonic diagnostic gel composition which is obtained by gelling the ultrasonic transmission efficiency improving composition; and an imaging method using the ultrasonic transmission efficiency improving composition and the ultrasonic diagnostic gel composition.

### TECHNICAL FIELD

The present invention relates to an ultrasonic transmission efficiency improving composition containing a component for improving ultrasonic transmission efficiency at the time of ultrasonic diagnosis, an ultrasonic diagnostic gel composition which is an application-type contact medium obtained by gelling the ultrasonic transmission efficiency improving composition, and an ultrasonic imaging method using the ultrasonic transmission efficiency improving composition and the ultrasonic diagnostic gel composition.

### BACKGROUND ART

Since the ultrasonic diagnosis is a non-invasive diagnosis, it is often used in health checkups. In the ultrasonic diagnosis of a living body, in order to ensure excellent acoustic propagation between the surface of the living body and transmitting and receiving surfaces of an ultrasonic probe, an application-type contact medium for ultrasonic diagnosis as an acoustic matching medium between those surfaces is introduced. It is usually an aqueous material (liquid substance) with a relatively high viscosity such as gel and gel. In normal ultrasonic diagnosis, firstly the application-type contact medium is applied to the skin, and ultrasonic waves are transmitted by pressing a probe to the skin with the medium applied to perform diagnosis using reflection of the ultrasonic waves on the subcutaneous tissues.

However, it is extremely difficult to obtain a clear image within a few centimeters below the surface due to the properties of the ultrasonic diagnostic device when the probe of the ultrasonic diagnostic device is directly pressed to the body surface to observe the internal state. In addition, in a case where a subject is a person who has many wrinkles formed on the skin, or has increased non-uniform skin structure, such as an elderly person, it has been difficult to obtain a clear image even though such a gel-like contact medium is applied.

Patent Literature 1 discloses that as an application-type contact medium for ultrasonic diagnosis capable of efficiently transmitting the ultrasonic waves to the skin, a gel composition containing an alkyl-modified carboxyvinyl polymer and/or a salt thereof, a carboxyvinyl polymer and/or a salt thereof, hydroxyalkyl cellulose, polyacrylic acid and/or a salt thereof, and a polyhydric alcohol is used. Further, as for the polyhydric alcohol, 1,3-butylene glycol, 1,2-pentanediol, 1,3-pentanediol, glycerin, sorbitol, mannitol, diglycerin, polyglycerin, ethylene glycol, diethylene glycol, polyethylene glycol, propylene glycol, dipropylene glycol, and pentaerythritol are used alone or in combination of two or more of 10.0% to 50.0% by weight.

Patent Literature 2 discloses an application-type contact medium for ultrasonic diagnosis, which is made of a gel containing curdlan as a main component and in which at least a portion of the gel is chemically crosslinked. It is also disclosed that the application-type contact medium for ultrasonic diagnosis has appropriate flexibility, mechanical strength, and excellent acoustic properties (such as a low ultrasonic attenuation rate).

Patent Literature 3 discloses that an application-type contact medium for ultrasonic diagnosis containing a (meth)acrylic acid polymer or a salt thereof, a (meth)acrylic acid/(meth)acrylic acid ester copolymer or a salt thereof, and a polyhydric alcohol having 3 to 6 hydroxyl groups in a molecule produces a flattering ultrasonic image while maintaining a practical viscosity with which the medium does not flow on the surface of a living body, is easy to squeeze from a container, easy to stretch, is non-sticky, salt-resistant, and is not solidified after drying.

Patent Literature 4 discloses an application-type contact medium for ultrasonic diagnosis that includes a salt of a carboxyvinyl polymer and xanthan gum, and further contains a polyhydric alcohol at a high concentration, so that a clear image can be obtained even in an elderly person whose clear image has not been obtainable.

Patent Literature 5 discloses that a contact medium for ultrasonic diagnosis that contains alginate in a solid matrix polymer of a polymerizable vinyl monomer is a hardly dryable and smooth solid which has high ultrasonic conduction efficiency not to adversely affect images (specifically, acoustic impedance is close to a living body and attenuation of ultrasonic waves is small), is highly resistant to sweat and temperature, does not cause skin allergies, and does not have a foul odor.

Patent Literature 6 discloses a technique for improving ultrasonic transmission sensitivity using ethylene glycol, diethylene glycol, triethylene glycol, tetraethylene glycol, 1,2-propanediol, 1,3-propanediol, glycerin, 1,2-butanediol, 1,3-butanediol, 1,4-butanediol, and 1,5-pentanediol, as an ultrasonic transmission medium, in order to prevent a decrease in sensitivity due to reflection of an echo signal resulting from a difference in acoustic impedance between an ultrasonic probe and an application-type contact medium for ultrasonic diagnosis.

Patent Literature 7 discloses that if an application-type contact medium for ultrasonic diagnosis contains an emollient component that imparts flexibility and moisture to the skin, transmission of ultrasonic waves is improved, sliding between the skin and a device is made smooth, stickiness after use is little, actual safety for the skin and a safety image are excellent, and an appropriate cosmetic effect is obtained.

However, such an application-type contact medium for ultrasonic diagnosis is still insufficient in the ultrasonic transmission sensitivity at the time of actual diagnosis. In other words, methods for improving physical properties such as gel strength of the application-type contact medium for ultrasonic diagnosis and preventing a decrease in sensitivity between the probe and the application-type contact medium for ultrasonic diagnosis have been performed; however, studies on sensitivity reduction in a stratum corneum of the skin which is a measurement site have not been performed, and improvement in diagnostic sensitivity is insufficient.

The stratum corneum of the skin is composed of stratum corneum cells and intercellular lipids. The intercellular lipid has a lipid lamellar structure and is moistened with moisture. However, people who have rough skin are in a state where the lamellar structure is disturbed and moisture easily evaporates. In addition, the elderly experiences a stratum corneum deposition phenomenon in which a peeling enzyme does not work due to aging and thereby a horny layer accumulates, and thus the horny layer accumulation prevents hydration from the lower skin to the upper skin, resulting in the dry upper skin.

In addition, a dry skin constitution has a phenomenon that NMF (natural moisturizing factor) components are insufficient and the horny layers are insufficient in the flexibility and moisture content. In this way, when the moisture content in the stratum corneum is small, there is a problem in that ultrasonic waves are refracted at the interface of paths with different velocities, reflected between media with large acoustic impedance differences, or scattered when the ultrasonic waves are incident on boundaries with sufficiently small acoustic impedance differences, and thereby sensitivity is lowered.

### CITATIONS LIST

### Patent Literature

Patent Literature 1: Japanese Patent No. 5576165
Patent Literature 2: Japanese Patent No. 3183583
Patent Literature 3: Japanese Patent No. 4358020
Patent Literature 4: Japanese Unexamined Patent Application Publication No. H11-318898
Patent Literature 5: Japanese Patent No. 2786465
Patent Literature 6: Japanese Unexamined Patent Application Publication No. 2006-198162
Patent Literature 7: Japanese Patent No. 4104720

### SUMMARY OF INVENTION

### Technical Problems

The present invention has been made in view of the above-described conventional situation, and particularly, an object thereof is to provide an ultrasonic transmission efficiency improving composition that improves ultrasonic transmission efficiency by suppressing a decrease in sensitivity at the time of ultrasonic diagnosis in a stratum corneum at a measurement site, a ultrasonic diagnostic gel composition that is an application-type contact medium obtained by gelling the ultrasonic transmission efficiency improving composition, and an imaging method using the ultrasonic transmission efficiency improving composition and the ultrasonic diagnostic gel composition.

### Solutions to Problems

As a result of verifying the prior art to solve the above-mentioned problems, the inventors of the present invention have recognized that, as described above, methods for improving physical properties such as gel strength of the application-type contact medium for ultrasonic diagnosis and preventing a decrease in sensitivity between the probe and the application-type contact medium for ultrasonic diagnosis have been performed; however, studies on sensitivity reduction in a stratum corneum which is a measurement site have not been performed, and improvement in diagnostic sensitivity is insufficient, and they have intensively studied measures to reduce sensitivity in the stratum corneum. That is, since the ultrasonic waves are easily transmitted in water and have little attenuation, when the moisture content of the application-type contact medium is maintained high, and the moisture content of the stratum corneum is increased at the measurement site, attenuation when the ultrasonic waves pass through the stratum corneum is suppressed, and thereby the ultrasonic waves can reach a deeper part in the living body. As a result, a reflected wave with higher intensity can be detected from a deeper portion in the living body.

Patent Literature 7 discloses an application-type contact medium for ultrasonic diagnosis containing an oil agent such as cacao oil or shea oil as an emollient component that gives skin flexibility and moisture; however, although the oil agent exists on the skin surface, and suppresses the evaporation of moisture from the skin, it does not replenish the stratum corneum with sufficient moisture, and an effect thereof is limited.

As a result of searching for a component that actively replenishes the skin stratum corneum with moisture, the inventors of the present invention have found that a component that forms a vesicle, which is a vesicle having a membrane structure and containing an aqueous phase in water can transport sufficient moisture to the stratum corneum, and have completed the invention disclosed in Japanese Patent No. 6130538.

On the other hand, since the application-type contact medium for ultrasonic diagnosis may be used at room temperature or may be used by heating to about 40 degrees, the inventors of the present invention have further searched for a component in which the above effect is stable even for this temperature change (temperature cycle). As a result, it has been found that glycogen and dextrin have an effect of actively transporting and replenishing moisture to the skin stratum corneum, and the effect is stable even after a temperature cycle when using an application-type contact medium for ultrasonic diagnosis, and thereby the ultrasonic transmission efficiency improving composition of the present invention has been completed. Moreover, the ultrasonic diagnostic gel composition as the application-type contact medium for ultrasonic diagnosis has been completed by adding a gelling agent or the like to the ultrasonic transmission efficiency improving composition to make it gelatinous.

Further, the inventors have found that when a vesicle forming component and an Alcaligenes-produced polysaccharide are allowed to coexist in glycogen and/or dextrin, there is a synergistic effect on the effect of actively transporting and replenishing moisture to the skin stratum corneum, and the effect can be maintained even through a temperature cycle, and have completed the present invention.

That is, the invention according to claim 1 is an ultrasonic transmission efficiency improving composition including an aqueous solution containing hydrophilic fine particles including glycogen and/or hydrophilic fine particles including dextrin.

The glycogen used in the invention according to claim 1 is known as a storage polysaccharide mainly in animals, and is a polymer having a network structure in which D-glucose is polymerized by α-glycoside bonds and has many branches. As natural glycogen derived from animals, glycogen obtained from bovine liver, shellfish meat of oyster and Mediterranean mussel, and the like is commercially available, phytoglycogen present in plants such as corn, and enzyme-synthesized glycogen biochemically synthesized from plant starch is also known.

The dextrin used in the invention according to claim 1 is a low molecular weight polysaccharide obtained by hydrolysis of starch or glycogen, in which D-glucose is polymerized by α-glycoside bonds. Dextrin having a cyclic structure is called cyclic dextrin.

The hydrophilic fine particles in the invention according to claim 1 are fine particles that are dispersed in water by themselves only by stirring or the like without being affected by other compounds such as a surfactant.

The invention according to claim 2 is the ultrasonic transmission efficiency improving composition according to claim 1, wherein the glycogen is one or more selected from the group consisting of mussel glycogen, phytoglycogen, and enzyme-synthesized glycogen.

The mussel glycogen used in the invention according to claim 2 is glycogen obtained from shellfish meat of Mediterranean mussel, phytoglycogen is glycogen obtained from a plant such as corn, and the enzyme-synthesized glycogen is biochemically synthesized from plant starch.

The invention according to claim 3 is the ultrasonic transmission efficiency improving composition according to claim 1 or claim 2, wherein the dextrin is cyclic dextrin.

The cyclic dextrin used in the invention according to claim 3 is polysaccharides having a cyclic structure in which D-glucose is bonded by an α-glycoside bond, and contains highly branched cyclic dextrin and the like.

The invention according to claim 4 is the ultrasonic transmission efficiency improving composition according to any one of claims 1 to 3, wherein the hydrophilic fine particles are contained in an amount of 0.005% by weight to 3% by weight with respect to a total amount of the aqueous solution.

In the invention according to claim 4, the hydrophilic fine particles are contained in an amount of 0.005% by weight to 3% by weight with respect to the total amount of the aqueous solution, and among these, 0.05% by weight or more and 0.25% or less are preferable.

In a case where the content of the hydrophilic fine particles is less than 0.005% by weight with respect to the total amount of the aqueous solution, the number of particles in the aqueous solution is small, and thus the effect of transporting and replenishing moisture to the skin stratum corneum may be reduced, and in a case where the content of the hydrophilic fine particles exceeds 3% by weight with respect to the total amount of the aqueous solution, the above effect may not be improved enough to satisfy the increase in content, and further, since aggregation of the hydrophilic fine particles in water tends to occur, the number of particles that can enter the skin stratum corneum may be reduced.

The invention according to claim 5 is the ultrasonic transmission efficiency improving composition according to any one of claims 1 to 4, wherein a particle size of the hydrophilic fine particle is in a range of 0.01 μm to 2.0 μm.

If the particle size of the hydrophilic fine particles in the present invention is in the range of 0.01 μm to 2.0 μm, moisture can be efficiently transported and replenished to the skin stratum corneum, and among these, the preferable particle size range is 0.01 μm to 1 μm, and the more preferable particle size range is 0.02 μm to 0.5 μm.

In a case where the particle size is less than 0.01 μm, the mass of the particles is small, so that the effect of transporting and replenishing moisture may be reduced, and in a case where the particle size exceeds 2.0 μm, the size of the particles is large, so that it is difficult to move freely in the intercellular lipids, and as a result, the effect of transporting and replenishing moisture may be reduced.

The invention according to claim 6 is the ultrasonic transmission efficiency improving composition according to any one of claims 1 to 5, wherein the aqueous solution contains an Alcaligenes-produced polysaccharide.

Since the Alcaligenes-produced polysaccharide used in the invention according to claim 6 contains at least a polysaccharide represented by the following General Formula (1), the aqueous solution containing the Alcaligenes-produced polysaccharide is excellent in water retention and the ultrasonic transmission efficiency improving composition excellent in stable viscosity over time can be obtained, and since viscosity and moisture can be maintained even during the use of ultrasonic diagnosis, the transmission efficiency of ultrasonic waves can be improved, and the sensitivity during the ultrasonic diagnosis can be maintained.

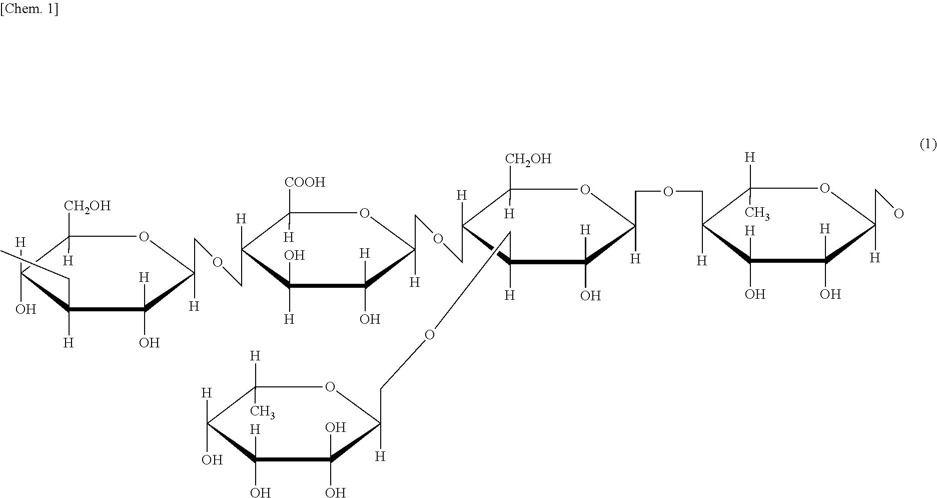

The invention according to claim 7 is the ultrasonic transmission efficiency improving composition according to any one of claims 1 to 6, wherein the aqueous solution contains a vesicle forming component that is a compound that forms a vesicle in water.

Examples of the vesicle forming component used in the invention according to claim 7 include sugar fatty acid ester, polyoxyethylene hydrogenated castor oil derivative, polyglycerol fatty acid ester, phospholipid, a phospholipid polymer, a mannosyl erythritol lipid, an acylamino acid metal salt, and ceramide, and these compounds can form vesicles rapidly by stirring with water, and can retain water in the vesicle structure and supply water to the stratum corneum of the skin.

The invention according to claim 8 is an ultrasonic diagnostic gel composition including the ultrasonic transmission efficiency improving composition according to any one of claims 1 to 7, and a carboxyvinyl polymer and/or a (meth)acrylic acid/(meth)acrylic acid ester copolymer and a salt thereof in a gel state.

The carboxyvinyl polymer and the (meth)acrylic acid/(meth)acrylic acid ester copolymer and a salt thereof used in the invention according to claim 8 are gelling agents that make the ultrasonic transmission efficiency improving composition into a gel state. By making the ultrasonic transmission efficiency improving composition of the present invention into a gel state, an ultrasonic diagnostic gel composition as an application-type contact medium for ultrasonic diagnosis can be obtained, and by performing the ultrasonic diagnosis after applying the ultrasonic diagnostic gel composition to the skin of the subject, water can be replenished to the skin stratum corneum and the ultrasonic wave can reach a deeper portion in the living body. This ultrasonic diagnostic gel composition is used in a second embodiment described later.

The invention according to claim 9 is the ultrasonic diagnostic gel composition according to claim 8, wherein a mixing amount of the carboxyvinyl polymer is 0.05% by weight to 5.0% by weight with respect to the total amount of the ultrasonic diagnostic gel composition, and a mixing amount of the (meth)acrylic acid/(meth)acrylic acid ester copolymer and the salt thereof is 0.05% by weight to 5.0% by weight with respect to the total amount of the ultrasonic diagnostic gel composition.

In the invention according to claim 9, an optimum mixing amount range of the carboxyvinyl polymer, and the (meth)acrylic acid/(meth)acrylic acid ester copolymer and the salt thereof with respect to the total amount of the ultrasonic diagnostic gel composition is defined, and when being in this range, the ultrasonic diagnostic gel composition is prevented from sagging from the skin, and between the ultrasonic probe and the skin, the ultrasonic transmission efficiency improving composition is prevented from being discharged from a contact surface by the pressure applied at the time of measurement, so that the workability is improved and the usability is excellent.

On the other hand, when the mixing amount of the carboxyvinyl polymer is less than 0.05% by weight with respect to the total amount of the ultrasonic diagnostic gel composition, a structure as the gel composition may not be formed, and when the mixing amount exceeds 5% by weight, the gel structure becomes excessively hard and may cause problems in use feeling. In addition, when the mixing amount of the (meth)acrylic acid/(meth)acrylic acid ester copolymer is less than 0.01% by weight, a structure as the gel composition may not be formed, and when the mixing amount exceeds 5% by weight, the gel structure becomes excessively hard and may cause problems in use feeling or the transparency of the gel may be lowered.

The invention according to claim 10 is an ultrasonic imaging method including: applying the ultrasonic transmission efficiency improving composition according to any one of claims 1 to 7 to a skin; and thereafter, applying an ultrasonic diagnostic gel composition that does not contain the ultrasonic transmission efficiency improving composition as it is to perform ultrasonic imaging, or thereafter, wiping off the ultrasonic transmission efficiency improving composition and applying the ultrasonic diagnostic gel composition that does not contain the ultrasonic transmission efficiency improving composition to perform ultrasonic imaging.

There are mainly two embodiments of the ultrasonic imaging method using the ultrasonic transmission efficiency improving composition of the present invention. The invention according to claim 10 prescribes the first embodiment, in which firstly the ultrasonic transmission efficiency improving composition of the present invention is applied to the skin of the subject to replenish sufficient moisture to the stratum corneum of the skin, and then the ultrasonic diagnostic gel composition that does not contain the ultrasonic transmission efficiency improving composition is applied to perform ultrasonic imaging. In this embodiment, there are further two methods. One is to apply the ultrasonic diagnostic gel composition not containing the ultrasonic transmission efficiency improving composition as it is after applying the ultrasonic transmission efficiency improving composition of the present invention to the skin of the subject, and the other one is to apply the ultrasonic diagnostic gel composition not containing the ultrasonic transmission efficiency improving composition after wiping off the ultrasonic transmission efficiency improving composition. According to these two methods, in both methods, sufficient moisture can be replenished to the stratum corneum of the skin, and thus the ultrasonic transmission efficiency can be improved to realize a highly sensitive ultrasonic imaging method.

The invention according to claim 11 is the ultrasonic imaging method wherein the ultrasonic diagnostic gel composition according to claim 8 or claim 9 is applied to the skin, and then ultrasonic imaging is performed as it is.

The invention according to claim 11 defines the second embodiment of the ultrasonic imaging method using the ultrasonic transmission efficiency improving composition of the present invention, and uses the ultrasonic diagnostic gel composition obtained by adding a gelling agent to the ultrasonic transmission efficiency improving composition of the present invention to be in a gel state. The ultrasonic diagnosis is performed after applying the ultrasonic diagnostic gel composition to the skin of the subject, so that water is replenished to the skin stratum corneum and ultrasonic transmission efficiency is improved to enable a highly sensitive ultrasonic imaging method.

### Advantageous Effects of Invention

Before applying the application-type contact medium for ultrasonic imaging to the skin, by applying the ultrasonic transmission efficiency improving composition of the present invention to the skin, and applying to the skin the ultrasonic diagnostic gel composition obtained by adding a gelling agent or the like to the ultrasonic transmission efficiency improving composition to be in a gel state, the ultrasonic transmission efficiency at the time of ultrasonic diagnosis is improved, the ultrasonic captured image is sharpened, and deep parts of organs under the skin at a captured portion can be observed. Furthermore, by using the ultrasonic transmission efficiency improving composition and the ultrasonic diagnostic gel composition of the present invention, stable and excellent ultrasonic imaging can be performed even through a temperature cycle when using the application-type contact medium for ultrasonic diagnosis.

### BRIEF DESCRIPTION OF DRAWINGS

FIG. 1 illustrates comparative photographs of ultrasonic captured images of Example 40 and Comparative Example 7.

FIG. 2 illustrates comparative photographs of ultrasonic captured images of Example 40 and Comparative Example 6 before and after the lapse of 5 days of temperature history.

FIG. 3 illustrates comparative photographs of ultrasonic captured images of Example 41 and Comparative Example 6 before and after the lapse of 5 days of temperature history.

FIG. 4 is an ultrasonic captured image of Doppler display of Comparative Example 6.

FIG. 5 is an ultrasonic captured image of Doppler display of Example 40.

### DESCRIPTION OF EMBODIMENTS

The present invention relates to an ultrasonic transmission efficiency improving composition containing a component for improving ultrasonic transmission efficiency at the time of ultrasonic diagnosis, an ultrasonic diagnostic gel composition which is an application-type contact medium obtained by gelling the ultrasonic transmission efficiency improving composition, and an ultrasonic imaging method using the ultrasonic transmission efficiency improving composition and the ultrasonic diagnostic gel composition. As a method for improving the ultrasonic transmission efficiency, attention was paid to supplying sufficient moisture to the stratum corneum of the skin at a measurement site.

That is, an object of the present invention is to search for a component capable of supplying sufficient moisture in the skin stratum corneum with low moisture, such as the skin of elderly people and dry skin, and as a substance having high water retention performance, a carboxyvinyl polymer which is a crosslinkable acrylic acid of a water absorbent resin is known; however, the inventors of the present invention have intensively searched for biological substances and substances derived from organism as components that actively transport and replenish moisture to the skin stratum corneum, and have found glycogen and dextrin. With this glycogen and dextrin, a stable hydration effect can be imparted to the ultrasonic transmission efficiency improving composition and the ultrasonic diagnostic gel composition even after a temperature cycle of 40° C. to room temperature repeated during ultrasonic diagnosis.

The reason why glycogen and dextrin have a remarkable effect on the object of the present invention is unclear, but a common point between the two is that glycogen and dextrin are each α-glucan in which D-glucose is linked by an α-glycoside bond. For example, xanthan gum, for which no particular effect was found in this search, is a microbially produced polysaccharide having glucose as the constituent monosaccharide like dextrin, but is not glucan, and cellulose is glucan but is not α-glucan. Therefore, it is presumed that the above-described common point suggests the reason why unique effects of the present invention are exhibited in glycogen and dextrin.

Examples of the glycogen used in the present invention include animal glycogen derived from shellfish such as scallops, abalone, oysters, mussels, and pearl oysters, and livers of cows, pigs, and the like, plant glycogen derived from corn, barley, rice, potato, tapioca, and the like, and if necessary, glycogen separated and purified after an enzyme treatment, specific examples of animal glycogen include biosaccharide LS and biosaccharide GY, which are Mediterranean mussel-derived mussel glycogens produced by LABORATORIES SEROBIOLOGIQUES, specific examples of plant glycogen include phytoglycogen (average molecular weight 700,000) produced by Kewpie Corporation, and specific examples of the glycogen separated and purified after the enzyme treatment include bioglycogen (shape is a spherical nanomolecule having a particle size of 20 to 60 nm) produced by Glico Foods Co., Ltd., biochemically synthesized from plant starch as a raw material by the action of enzymes.

The dextrin used in the present invention is obtained by hydrolysis of starch or glycogen, and a structure thereof is a polymer in which a large number of D-glucoses are polymerized by α-glycoside bonds (α1→4 bonds and α1→6 bonds) to form a highly branched structure, and may have a cyclic structure. Specific examples thereof include AMYCOL No. 10 produced by Nippon Starch Chemical Co., Ltd. and CLUSTER DEXTRIN (highly branched cyclic dextrin) produced by Glico Foods Co., Ltd. and the like.

The Alcaligenes-produced polysaccharide used in the present invention belongs to polysaccharides containing at least a polysaccharide represented by the following General Formula (2), and this polysaccharide is marketed by Hakuto Co., Ltd under the trade name “ALCASEALAN”, “Cosmetics display name Alcalinegenes-produced polysaccharide”.

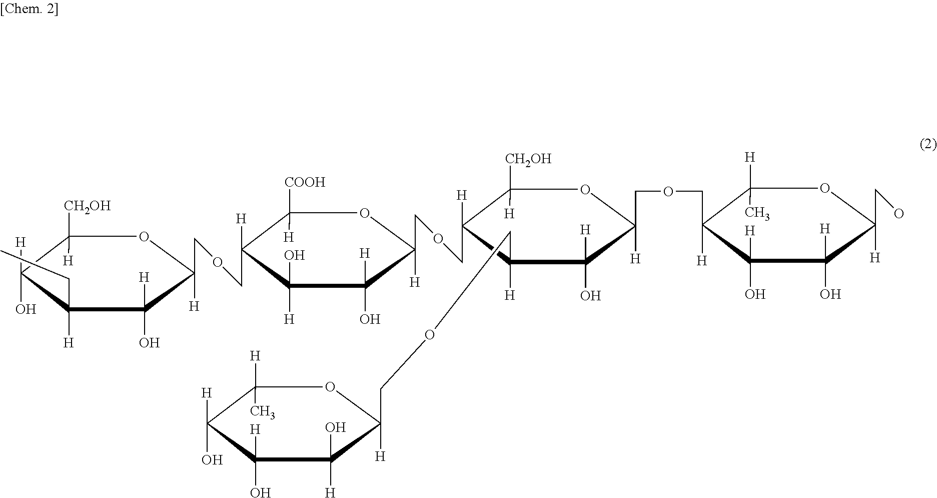

Japanese Patent No. 4902212 discloses that the Alcaligenes-produced polysaccharide is present as particles having an average particle size of 8 nm to 500 nm in an aqueous solution, and although the polysaccharide has a bond other than an α-glycoside bond, it is presumed to have an effect of assisting the effect of the hydrophilic fine particles of the present invention from the fact that it has excellent moisture retention and is formed into a hydrophilic particle state in water.

Although the vesicle forming component used in the present invention forms a vesicle in water, as described above, the vesicle alone cannot maintain the form after a temperature cycle. However, other than that, it has the same effect as the hydrophilic fine particles of the present invention. In a case where the hydrophilic fine particles and vesicles coexist in an aqueous solution, since different compounds have the same effect and are dispersed while balancing attraction and repulsion with each other, the probability that the same type of particles are associated and aggregated is decreased, and therefore, the hydrophilic fine particles of the present invention maintain an excellent dispersion state in an aqueous solution, and can stably exhibit the effect of transporting and replenishing the moisture to the skin stratum corneum.

Furthermore, in a case where the hydrophilic fine particles of the present invention and the vesicle coexist in an aqueous solution, a unique effect that the collapse of the vesicle form due to temperature change is suppressed is obtained, which cannot be obtained with vesicles alone.

As the vesicle forming component, compounds such as sugar fatty acid ester, a polyoxyethylene hydrogenated castor oil derivative, polyglycerol fatty acid ester, a phospholipid, a phospholipid polymer, a mannosyl erythritol lipid, an acylamino acid metal salt, ceramide, and the like can be used.

Examples of the sugar fatty acid ester include sucrose fatty acid ester, maltitol fatty acid ester, and trehalose fatty acid ester. The number of hydroxyl groups substituted with fatty acid (degree of esterification) is not particularly limited, but monoesters, diesters and triesters are preferable, monoesters and diesters are more preferable, and monoesters are most preferable. The constituent fatty acid in the sugar fatty acid ester is saturated or unsaturated fatty acid having 12 to 22 carbon atoms, and preferably has a straight chain or a branched chain.

Examples of these fatty acids include lauric acid, myristic acid, palmitic acid, stearic acid, isostearic acid, arachic acid, behenic acid, tetradecenoic acid, hexadecenoic acid, octadecenoic acid, octadecadienoic acid, eicosenoic acid, eicosatetraenoic acid, docosenoic acid, and octadecatrienoic acid. Among these, stearic acid is preferable. In the case of a diester, two fatty acids may be different from each other.

The polyoxyethylene hydrogenated castor oil derivative is produced by hydrogenating castor oil and subjecting ethylene oxide to addition polymerization. Although HLB varies depending on the number of ethylene oxide, HLB 10 to 15 is preferable and HLB 11 to 14 is more preferable for the purpose of the present invention.

The polyglycerol fatty acid ester is formed by ester bonding of polyglycerin and fatty acid, but the type of fatty acid is not particularly limited. For example, it can be obtained by hydrolyzing and purifying oil extracted from natural animals and plants with separation or without separation, may be saturated, unsaturated, or a mixture of both, and is preferably saturated fatty acid, more preferably at least one selected from the group consisting of myristic acid, palmitic acid, and stearic acid, and most preferably stearic acid.

The degree of polymerization of the polyglycerol constituting the polyglycerol fatty acid ester is not particularly limited, but the average degree of polymerization is 2 to 20, preferably 4 to 10, and more preferably 5 to 10.

Further, although not particularly limited, the polyglycerol fatty acid ester has a molar average esterification degree of preferably 3.0 or less, more preferably 2.0 or less, and most preferably 1.5 or less. In a case where the purity is 100%, the molar average esterification degree is 1 for monoester and 2 for diester.

The molar average esterification degree of the mixture is a weighted average of the molar average esterification degree of each of the mixed components using a mixing weight ratio. For example, in a case where the polyglycerol fatty acid ester having a degree of esterification of 1 is 60% by weight and the polyglycerol fatty acid ester having a degree of esterification of 2 is 40% by weight, an average degree of esterification is 1.4 of the weighted average thereof.

Further, the HLB of the polyglycerol fatty acid ester in the present invention is preferably HLB 9 to 16, and more preferably HLB 11 to 14.

Preferable examples of the phospholipid include those selected from lecithin, phosphatidylglycerol, phosphatidylserine, phosphatidylinositol, phosphatidylethanolamine, phosphatidylic acid, and lyso forms thereof. Here, lecithin is a common name for phospholipids containing phosphatidylcholine as a main component, and instead of lecithin, phosphatidylcholine as a main component can also be used. As the base source, soybean, egg yolk, and the like can be preferably exemplified, and soybean can be particularly preferably exemplified. In the vesicle forming compound used in the present invention, one or more phospholipids can be used.

Examples of the phospholipid polymer include 2-methacryloyloxyethyl phosphorylcholine represented by the following General Formula (3), and examples of a commercially available product of this compound include LIPIDURE-NR (produced by NOF CORPORATION).

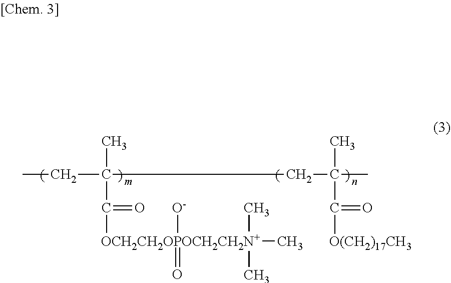

Mannosyl erythritol lipid is a compound produced by yeast, and is a compound having a structure in which erythritol is substituted at the 1-position of mannose, acyl groups are substituted at the 2- and 3-positions, and acetyl groups are substituted at the 4- and 6-positions. Examples of a commercial product include CERAMELA-PX (produced by TOYOBO CO., LTD.).

Examples of the acyl amino acid metal salt include sodium N-lauroyl-L glutamate, sodium N-stearoyl-L glutamate, sodium di(N-lauroylglutamyl) lysine, and sodium lysine dilauroylglutamate. Examples of a commercially available product include PELLICER L-30 (produced by Asahi Kasei Corporation.).

There are ceramide types 1 to 7 depending on the structure thereof. Among these, ceramide 2 and ceramide 3 can be exemplified as more preferable ones, and ceramide 2 is particularly preferable. Such ceramide is commercially available as a raw material for an external preparation for skin, and in the present invention, such a commercial product can be purchased and used.

Examples of the ceramide as a commercially available cosmetic raw material include “Ceramide 2” (ceramide 2) (produced by COSMO FARM.), “Ceramide III” (ceramide 3) (produced by COSMO FARM.), “Ceramide IIIA” (ceramide 3)) (produced by COSMO FARM.), “Ceramide IIIB” (ceramide 3), “Ceramide VI” (ceramide 6) (produced by COSMO FARM.), “Ceramide TTI-001” (ceramide 2) (produced by Takasago International Corporation), Sphingomonas ferment extract, cerebroside, and sphingomyelin.

The ultrasonic diagnostic gel composition of the present invention is produced as a gel-like form by adding a gelling agent to the ultrasonic transmission efficiency improving composition. In the above-described first embodiment in which the ultrasonic transmission efficiency improving composition of the present invention is applied to the skin of the subject before applying the application-type contact medium for ultrasonic diagnosis to the skin, the application operation is performed twice, which is laborious. Therefore, in the above-described second embodiment, the ultrasonic diagnostic efficiency gel composition of the present invention is produced by making the ultrasonic transmission efficiency improving composition into a gel-like form so as to produce an ultrasonic diagnostic gel composition into a single solution, and the application operation is performed once, thereby simplifying the operation and improving the practicality. The ultrasonic diagnostic gel composition of the present invention is used in this second embodiment.

Examples of the gelling agent used in the ultrasonic diagnostic gel composition of the present invention include a carboxyvinyl polymer, a (meth)acrylic acid/(meth)acrylic acid ester copolymer and a salt thereof. The carboxyvinyl polymer is mainly a polymer of acrylic acid, and specific examples thereof include HIVISWAKO 103 (produced by Wako Pure Chemical Industries, Ltd.), HIVISWAKO 104 (produced by Wako Pure Chemical Industries, Ltd.), HIVISWAKO 105 (produced by Wako Pure Chemical Industries, Ltd.n), AQUPEC HV-501E (produced by Sumitomo Seika Chemicals Company, Limited), AQUPEC HV-805EG (produced by Sumitomo Seika Chemicals Company, Limited), AQUPEC HV-504E (produced by Sumitomo Seika Chemicals Company, Limited), AQUPEC HV-505E (produced by Sumitomo Seika Chemicals Company, Limited), Carbopol 981 (produced by The Lubrizol Corporation), Carbopol 980 (produced by The Lubrizol Corporation), Carbopol 941 (produced by The Lubrizol Corporation), Carbopol 940 (produced by The Lubrizol Corporation), Carbopol Ultrez 10 (produced by The Lubrizol Corporation), Carbopol 2984 (produced by The Lubrizol Corporation), and Carbopol ETD2050 (produced by The Lubrizol Corporation), and one or more of these can be appropriately selected to be used.

The carboxyvinyl polymer is usually used after neutralization with a basic substance. Examples of the basic substance include alkanolamines such as triethanolamine and monoethanolamine, inorganic bases such as ammonia, sodium hydroxide, and potassium hydroxide, and basic amino acids such as arginine. The basic substance is added in an amount sufficient to neutralize the carboxyvinyl polymer component, and the above components may be appropriately mixed and used.

The (meth)acrylic acid/(meth)acrylic acid alkyl copolymer used in the ultrasonic diagnostic gel composition of the present invention is a copolymer of at least one monomer composed of acrylic acid, methacrylic acid or a simple ester thereof, and acrylic acid alkyl. This copolymer is known as an INCI name: Acrylates/C10-30 Alkyl Acrylate Crosspolymer [(acrylic acid/acrylic acid (C10-30) alkyl) crosspolymer] and the like, and specific examples of a commercially available product include “Carbopol ETD2020 polymer”, “Carbopol 1342 polymer”, “Carbopol 1382 polymer”, “Carbopol Ultrez20 polymer”, “Carbopol Ultrez21 polymer”, “Pemulen TR-1”, “Pemulen TR-2” (which are produced by The Lubrizol Corporation), and AQUPEC HV-803ERK (produced by Sumitomo Seika Chemicals Company, Limited), and one or more of these can be appropriately selected to be used.

The (meth)acrylic acid/(meth)acrylic acid alkyl copolymer component is usually used after neutralization with a basic substance. Examples of the basic substance include alkanolamines such as triethanolamine and monoethanolamine, inorganic bases such as ammonia, sodium hydroxide, and potassium hydroxide, and basic amino acids such as arginine. The basic substance is added in an amount sufficient to neutralize the (meth)acrylic acid/(meth)acrylic acid alkyl copolymer component, and the above components may be appropriately mixed and used.

Other components that can be contained in the ultrasonic transmission efficiency improving composition of the present invention include polyhydric alcohols. The polyhydric alcohol is mixed such that an ultrasonic probe that comes in contact with the ultrasonic transmission efficiency improving composition and the ultrasonic diagnostic gel composition during ultrasonic diagnosis can be easily cleaned after measurement, and for the purpose of improving the familiarity with the skin. As the polyhydric alcohol, ethylene glycol, diethylene glycol, triethylene glycol, tetraethylene glycol, 1,3-propanediol, glycerin, 1,2-butanediol, 1,3-butanediol, 1,4-butanediol, 1, 5-pentanediol, 1,2-pentanediol, 1,3-pentanediol, sorbitol, mannitol, diglycerin, polyglycerin, ethylene glycol, diethylene glycol, polyethylene glycol, propylene glycol, dipropylene glycol, pentaerythritol, and the like can be mixed, and it is desirable to mix 5.0% to 50.0% by weight of one or more of 1,3-propanediol, glycerin, diglycerin, 1,3-butanediol, 1,2-pentanediol, and propylene glycol with respect to the ultrasonic transmission efficiency improving composition or the ultrasonic diagnostic gel composition, and it is more desirable to mix 10.0% to 30.0% by weight thereof.

Moreover, other components that can be contained in the ultrasonic transmission efficiency improving composition of the present invention include a thickener. Examples of the thickener include plant polymers such as gum arabic, tragacanth, galactan, carob gum, guar gum, caraya gum, carrageenan, pectin, agar, algae colloid, fucoidan, trant gum, locust bean gum, and galactomannan; microbial polymers such as xanthan gum, curdlan, gellan gum, fucogel, dextran, succinoglucan, pullulan, and diutan gum; animal polymers such as chitosan, casein, albumin, gelatin, collagen, and degraded collagen; starch polymers such as starch, carboxymethyl starch, and methylhydroxypropyl starch; cellulose polymers such as methylcellulose, ethylcellulose, methylhydroxypropylcellulose, carboxymethylcellulose, hydroxymethylcellulose, hydroxypropylcellulose, nitrocellulose, sodium cellulose sulfate, sodium carboxymethylcellulose, crystalline cellulose, and cellulose powder; alginate polymers such as sodium alginate and propylene glycol alginate; synthetic polymers such as polyvinyl methyl ether, polyoxyalkylene, sodium polyacrylate, polyethyl acrylate, polyacrylamide, and polyethyleneimine; and inorganic water-soluble polymers such as bentonite, laponite, and hectorite.

Furthermore, as other components that can be contained in the ultrasonic transmission efficiency improving composition of the present invention, components that give the skin flexibility and moisture can be exemplified, and these components have the effect of smoothing the sliding of the probe on the skin. Therefore, workability at the time of ultrasonic diagnosis can be improved. Examples of these components include mucopolysaccharides such as hyaluronic acid, chondroitin sulfate, dermatan sulfate, heparan sulfate, heparin and keratan sulfate, or salts thereof; proteins such as collagen, degraded collagen, elastin, and keratin, derivatives thereof and salts thereof; sugars such as honey, erythritol, maltose, maltitol, xylitol, xylose, pentaerythritol, fructose, mannitol, sorbitol, inositol, trehalose, and glucose; urea, asparagine, aspartic acid, alanine, arginine, isoleucine, ortinin, glutamine, glycine, and glutamic acid, derivatives thereof and salts thereof; cysteine, cystine, citrulline, threonine, serine, tyrosine, tryptophan, theanine, valine, histidine, hydroxylysine, hydroxyproline, pyrrolidone carboxylic acids, and salts thereof; and amino acids such as proline, phenylalanine, methionine, and lysine and derivatives thereof or salts thereof. Ingredients that give the skin flexibility and moisture are mixed by appropriately selecting one or more components, and the mixing amount varies depending on the kind of the components and cannot be determined uniformly, but is usually 0.01 to 5%.

The method for preparing the ultrasonic transmission efficiency improving composition and the ultrasonic diagnostic gel composition of the present invention is not particularly limited, and the compositions are prepared by adding the respective components to water, and mixing under stirring. However, in a case of adding powder, the order of addition and the addition method are adjusted so as not to become “lumps”.

### Examples

The present invention will be described in more detail with reference to the following examples, but the present invention is not limited thereto.

1. Components Used in Examples and Comparative Examples

(1) Hydrophilic fine particle forming component of the present invention

A-1 Mussel glycogen, trade name “Biosaccharide GY” (produced by LABORATORIES SEROBIOLOGIQUES)]

A-2 Phytoglycogen [trade name “Phytoglycogen” (Kewpie Corporation)]

A-3 Enzyme-synthesized glycogen [trade name “Bioglycogen” (produced by Glico Foods Co., Ltd.)]

A-4 Enzyme-modified dextrin [trade name “AMYCOL No. 7-H” (Nippon Starch Chemical Co., Ltd.)]

A-5 Enzyme-modified dextrin [trade name “AMYCOL No. 6-H” (Nippon Starch Chemical Co., Ltd.)]

A-6 Highly branched cyclic dextrin [trade name “Cluster dextrin” (produced by Glico Foods Co., Ltd.)]

(2) Vesicle Forming Component

B-1 Polyoxyethylene hydrogenated castor oil [trade name “NIKKOL HCO-30” (HLB11) (produced by Nikko Chemicals Co., Ltd.)]

B-2 Polyoxyethylene hydrogenated castor oil [trade name “NIKKOL HCO-60” (HLB14) (produced by Nikko Chemicals Co., Ltd.)]

B-3 Sphingomonas ferment extract [trade name “BIOCERA G” (produced by Dainihon Kasei Co., Ltd.)]

B-4 Dilauroyl glutamic acid lysine NA [trade name “PELLICER L-30” (produced by Asahi Kasei Corporation.)]

B-5 Phospholipid polymer [trade name “LIPIDURE-NR” (produced by NOF CORPORATION)]

B-6 Mannosyl Erythritol Lipid [trade name “CERAMELA-PX” (produced by TOYOBO CO., LTD.)]

(3) Alcaligenes-Produced Polysaccharide

C-1 Alcaligenes polysaccharide (INCI name: Alcaligenes Polysaccharides) (trade name “Alcasealan” produced by Hakuto Co., Ltd.)

(4) Gelling Agent

D-1 Carboxyvinyl polymer [trade name HIVISWAKO 103 (produced by Wako Pure Chemical Industries, Ltd.)]

D-2 Carboxyvinyl polymer [trade name HIVISWAKO 105 (produced by Wako Pure Chemical Industries, Ltd.)]

D-3 Carboxyvinyl polymer [trade name: AQUPEC HV-805EG (produced by Sumitomo Seika Chemicals Company, Limited)]

D-4 (Meth)acrylic acid/(meth)acrylic acid alkyl copolymer (trade name: AQUPEC HV-803ERK (produced by Sumitomo Seika Chemicals Company, Limited))

(5) Polyhydric Alcohol Component

E-1 Glycerin [trade name “Glycerin S”, produced by Sakamoto Yakuhin kogyo Co., Ltd.]

E-2 1,3-propanediol (special grade reagent produced by Wako Pure Chemical Corporation)

E-3 1,3-butanediol (trade name “1,3BG”, produced by Daicel Corporation.)

E-4 Propylene glycol [trade name “Propylene glycol for cosmetics”, produced by ADEKA Corporation]

(6) Thickener

F-1 Xanthan gum [trade name “Keltrol CG”, produced by SANSHO Co., Ltd.]

F-2 Locust Bean Gum Genu Gum (produced by SANSHO Co., Ltd.)

F-3 Gellan gum Kelcogel CG (produced by SANSHO Co., Ltd.)

(7) Water Used in Examples and Comparative Examples

G-1 Purified water (Japanese Pharmacopoeia)

(8) Other Components

J-1 1,3-(2-ethylhexyloxy)propane-1,2-diol (trade name “SENSIVA SC 50 JP”, produced by Schulke&Mayr)

J-2 Methylparaben (=methyl paraoxybenzoate) (produced by Ueno Fine Chemicals Industry, Ltd)

(9) Conventional Application-Type Contact Medium (Used as a Comparative Example)

H-1 Echo Jelly [trade name, produced by Hitachi, Ltd.]

2. Preparation Method of Ultrasonic Transmission Efficiency Improving Composition and Ultrasonic Diagnostic Gel Composition

(1) Preparation Method of Ultrasonic Transmission Efficiency Improving Composition

200 g of purified water is put into a 500 ml beaker, the hydrophilic fine particle forming component of the present invention is added under stirring by a disperser (the Alcaligenes-produced polysaccharide is added at this stage in a case of mixing the Alcaligenes-produced polysaccharide), and stirred at 5000 rpm for 10 minutes to obtain a hydrophilic fine particle dispersion. A predetermined amount of other components are added to this hydrophilic fine particle dispersion, and the volume is made up to 300 g with purified water to obtain the ultrasonic transmission efficiency improving composition of the present invention.

(2) Preparation Method of Ultrasonic Diagnostic Gel Composition

200 g of purified water is put into a 500 ml beaker, the gelling agent of the present invention is added under stirring by a disperser (the Alcaligenes-produced polysaccharide is added at this stage in a case of mixing the Alcaligenes-produced polysaccharide), and stirred at 5000 rpm for 10 minutes, then a predetermined amount of the hydrophilic fine particle forming component of the present invention and other components are added, and the volume is made up to 180 g with purified water, followed by further stirring for 10 minutes. The obtained solution is adjusted to pH 6.5 to 7.0 with 1% KOH aqueous solution and then the volume is made up to 200 g with purified water to obtain the ultrasonic diagnostic gel composition of the present invention.

3. Method for Measuring Evaluation Item

The effects of the ultrasonic transmission efficiency improving composition and the ultrasonic diagnostic gel composition of the present invention are evaluated by the moisture content of the skin stratum corneum and the ultrasonic reflection signal intensity, and determined by the measurement of the average particle size of the hydrophilic fine particles and the ultrasonic image. In addition, although a sensory test was performed, a method used for the test will be described later.

(1) Method for Measuring Moisture Content of Skin Stratum Corneum

After performing ultrasonic diagnosis using the ultrasonic transmission efficiency improving composition and the ultrasonic diagnostic gel composition, the excess gel is wiped off with tissue paper, and a skin conductance value after 10 minutes was measured using a skin horny layer moisture content measuring device (trade name “SKICON-200EX”, manufactured by IBS Co., Ltd.). The proximity location on the skin was measured 5 times, and the average value was defined as the skin conductance value (unit: μS). The skin conductance value has a positive correlation with the skin surface horny layer moisture content, and the higher the conductance value, the greater the skin stratum corneum moisture content.

(2) Method for Measuring Ultrasonic Reflection Signal Intensity

(A) Measuring device

a. Transceiver ECHOMETR 1060 (manufactured by KARL DEUTSCH)

b. Vibrator DS12HB 1-6 (1 to 6 MHz) (manufactured by KARL DEUTSCH)

c. Standoff 2 mm thick matching layer

(B) Measurement location Lower leg or forearm

(C) Measuring method

The reflection signal intensity reflected from the skin surface was measured with the above measuring device. The greater the percentage of ultrasonic waves reflected from the skin surface, the greater the reflection signal intensity, and thus the smaller the reflection signal intensity, the more ultrasonic waves pass through the skin surface without reflecting off the skin surface and reach a deeper portion of the skin. The reflection signal intensity is represented by the abbreviation Vpp, and the unit is mV.

(3) Method for Measuring Average Particle Size of Hydrophilic Fine Particles

(A) Measuring device Concentrated particle size analyzer FPAR-1000 (manufactured by Otsuka Electronics Co., Ltd.)

(B) Measuring method

1 g of hydrophilic fine particle dispersion prepared by the above method and 99 g of purified water are weighed into a beaker and stirred with a magnetic stirrer for 1 hour. An appropriate amount of this solution is put into the measuring cell of the above measuring device, and a probe is attached to start the measurement. Regarding the same sample, the measurement was performed 3 times to calculate an average value. The unit is μm.

(4) Method for Capturing Ultrasonic Image

As a measuring device for an ultrasonic captured image, PROSOUND α7 (manufactured by Hitachi, Ltd.) was used as the ultrasonic imaging device, and UST-567 (manufactured by Hitachi Aloka Medical, Ltd) was used as a probe.

As an evaluation method, a cross-sectional image (B image) was drawn on a living body, and “overall luminance”, “tissue appearance”, and “multiple reflection (α1) directly under the probe” were compared. Moreover, when carrying out evaluation in more detail, comparison was made using Doppler display (color Doppler display, pulse Doppler display).

4. Embodiments and Methods for Evaluating Ultrasonic Transmission Efficiency Improving Composition and Ultrasonic Diagnostic Gel Composition

### (1) EMBODIMENTS

### (A) First Embodiment (1)

An ultrasonic transmission efficiency improving composition immersed in cotton is adhered to a measurement site of a subject for 3 minutes. After the lapse of 3 minutes, the surface is wiped off with tissue to remove moisture, then the conventional application-type contact medium H-1 not containing the ultrasonic transmission efficiency improving composition is applied and extended, and an ultrasonic measurement probe is applied to the skin to measure the reflection signal intensity of the ultrasonic wave by using the above-described measuring method. Also, an ultrasonic image is captured as necessary. Thereafter, the excess gel is wiped off with tissue paper, and the skin conductance value is measured after 10 minutes.

### (B) First Embodiment (2)

An ultrasonic transmission efficiency improving composition immersed in cotton is adhered to a measurement site of a subject for 3 minutes. The measurement is the same as that in the first embodiment (2) except that the conventional application-type contact medium H-1 not containing the ultrasonic transmission efficiency improving composition is applied without wiping off the surface with tissue to remove moisture after the lapse of 3 minutes.

### (C) Second Embodiment

After applying and extending an ultrasonic diagnostic gel composition to a measurement site of a subject, the reflection signal intensity of the ultrasonic wave is measured by applying an ultrasonic measurement probe to the skin by using the above-described measuring method. Also, an ultrasonic image is captured as necessary. Thereafter, the excess gel is wiped off with tissue paper, and the skin conductance value is measured after 10 minutes.

5. Evaluation Test and Results

Table 1 indicated the mixtures of the ultrasonic transmission efficiency improving composition and the ultrasonic diagnostic gel composition used in Test 1 and Test 2. Unless otherwise specified, the mixing amount is expressed as “% by weight” with respect to the system in which the component is mixed.

TABLE 1

| No. | 1 | 2 | 3 | 4 | 5 | 6 | 7 | 8 |
|---|---|---|---|---|---|---|---|---|
| A-1 | 0.25 | 0 | 0 | 0 | 0 | 0 | 0 | 0 |
| A-2 | 0 | 0.25 | 0 | 0 | 0 | 0 | 0 | 0 |
| A-3 | 0 | 0 | 0.25 | 0 | 0 | 0 | 0.25 | 0 |
| A-4 | 0 | 0 | 0 | 0.25 | 0 | 0 | 0 | 0 |
| A-5 | 0 | 0 | 0 | 0 | 0.25 | 0 | 0 | 0 |
| A-6 | 0 | 0 | 0 | 0 | 0 | 0.25 | 0 | 0 |
| D-1 | 0 | 0 | 0 | 0 | 0 | 0 | 0.25 | 0 |
| F-1 | 0 | 0 | 0 | 0 | 0 | 0 | 0 | 0.25 |
| G-1 | 99.75 | 99.75 | 99.75 | 99.75 | 99.75 | 99.75 | 99.5 | 99.75 |

(1) Test 1—Evaluation of Embodiments

Regarding the first embodiment (1) and the first embodiment (2), Mixture 3 (enzyme-synthesized glycogen mixture) and Mixture 8 (xanthan gum mixture) are compared, and for the second embodiment, Mixture 7 (enzyme-synthesized glycogen+carboxyvinyl polymer) and H-1 (echo jelly), a conventional application-type contact medium, were compared. The results were indicated in Table 2.

TABLE 2

| | Examples | | | Comparative examples | | |
|---|---|---|---|---|---|---|
| | No. | | | | | |
| | 1 | 2 | 3 | 1 | 2 | 3 |
| Embodiments | First | First | Second | First | First | Second |
| | embodiment | embodiment | embodiment | embodiment | embodiment | embodiment |
| | (1) | (2) | | (1) | (2) | |
| Ultrasonic | Mixture 3 | Mixture 3 | Unused | Mixture 8 | Mixture 8 | Unused |
| transmission | | | | | | |
| efficiency | | | | | | |
| improving | | | | | | |
| composition | | | | | | |
| With or without | Wipe off | Do not wipe | | Wipe off | Do not wipe | |
| wiping off | | off | | | off | |
| Ultrasonic | H-1 | H-1 | Mixture 7 | H-1 | H-1 | H-1 |
| diagnostic gel | | | | | | |
| composition | | | | | | |
| Conductance value | 461 | 462 | 465 | 127 | 131 | 118 |
| (μS) | | | | | | |
| Vpp(mV) | 0.21 | 0.20 | 0.20 | 0.29 | 0.29 | 0.31 |

From the results of Test 1, when the ultrasonic transmission efficiency improving composition and the ultrasonic diagnostic gel composition of the present invention were compared with the composition containing xanthan gum and the conventional application-type contact medium in all embodiments, it was indicated that the skin stratum corneum had a high moisture content and that ultrasonic waves reach deeper portions of the skin. Moreover, there was almost no difference in the effects by the difference in embodiments.

(2) Test 2—Effects of Various Glycogen and Dextrin

The effect of the ultrasonic transmission efficiency improving composition obtained by mixing various glycogen and dextrin was confirmed. The results were indicated in Table 3.

TABLE 3

| | Examples | | | | | |
|---|---|---|---|---|---|---|
| | No. | | | | | |
| | 4 | 5 | 1 | 6 | 7 | 8 |
| Embodiments | First | First | First | First | First | First |
| | embodiment | embodiment | embodiment | embodiment | embodiment | embodiment |
| | (1) | (1) | (1) | (1) | (1) | (1) |
| Ultrasonic | Mixture 1 | Mixture 2 | Mixture 3 | Mixture 4 | Mixture 5 | Mixture 6 |
| transmission | | | | | | |
| efficiency | | | | | | |
| improving | | | | | | |
| composition | | | | | | |
| Average | 0.669 | 0.075 | 0.137 | 0.016 | 1.382 | 0.283 |
| particle | | | | | | |
| size (μm) | | | | | | |
| With or | Wipe off | Wipe off | Wipe off | Wipe off | Wipe off | Wipe off |
| without | | | | | | |
| wiping off | | | | | | |
| Ultrasonic | H-1 | H-1 | H-1 | H-1 | H-1 | H-1 |
| diagnostic | | | | | | |
| gel | | | | | | |
| composition | | | | | | |
| Conductance | 404 | 446 | 461 | 417 | 392 | 455 |
| value (μS) | | | | | | |
| Vpp(mV) | 0.25 | 0.22 | 0.21 | 0.24 | 0.26 | 0.21 |

From the results of Test 2, it was indicated that regardless of the type of glycogen or dextrin to be mixed, the skin stratum corneum to which the ultrasonic transmission efficiency improving composition of the present invention was applied had a large moisture content, and the ultrasonic waves reached up to a deeper portion in the skin. Among glycogens, the ultrasonic transmission efficiency improving composition of Mixture 3 containing enzyme-synthesized glycogen was highly effective, and among dextrin, the ultrasonic transmission efficiency improving composition of Mixture 6 containing highly branched cyclic dextrin, which is cyclic dextrin, was highly effective. It was also indicated that the average particle size of the hydrophilic fine particles in the solution was preferably in the range of 0.01 μm to 2.0 μm.

(3) Test 3—Evaluation by Content of Hydrophilic Fine Particles

Table 4 indicated the mixtures of the ultrasonic transmission efficiency improving composition used in Test 3 in which the content of the hydrophilic fine particles was changed. The application results were indicated in Table 5.

TABLE 4

| No. | 9 | 10 | 3 | 11 | 12 | 13 |
|---|---|---|---|---|---|---|
| A-3 | 0.005 | 0.05 | 0.25 | 0.5 | 1.0 | 3.0 |
| G-1 | 99.995 | 99.95 | 99.75 | 99.5 | 99.0 | 97.0 |

TABLE 5

| | Examples | | | | | |
|---|---|---|---|---|---|---|
| | No. | | | | | |
| | 9 | 10 | 1 | 11 | 12 | 13 |
| Embodiments | First | First | First | First | First | First |
| | embodiment | embodiment | embodiment | embodiment | embodiment | embodiment |
| | (1) | (1) | (1) | (1) | (1) | (1) |
| Ultrasonic | Mixture 9 | Mixture 10 | Mixture 3 | Mixture 11 | Mixture 12 | Mixture 13 |
| transmission | | | | | | |
| efficiency | | | | | | |
| improving | | | | | | |
| composition | | | | | | |
| With or | Wipe off | Wipe off | Wipe off | Wipe off | Wipe off | Wipe off |
| without | | | | | | |
| wiping off | | | | | | |
| Ultrasonic | H-1 | H-1 | H-1 | H-1 | H-1 | H-1 |
| diagnostic gel | | | | | | |
| composition | | | | | | |
| Conductance | 394 | 456 | 461 | 448 | 453 | 450 |
| value (μS) | | | | | | |
| Vpp (mV) | 0.25 | 0.22 | 0.21 | 0.23 | 0.22 | 0.22 |

From the results of Test 3, it was indicated that the skin stratum corneum to which the ultrasonic transmission efficiency improving composition of the present invention containing 0.005% by weight to 3.0% by weight of the hydrophilic fine particles was applied had a large moisture content, and the ultrasonic waves reached up to a deeper portion in the skin.

(4) Test 4-Mixing Effect of Vesicle Forming Component and Alcaligenes-Produced Polysaccharide

Table 6 indicated the mixtures of the ultrasonic transmission efficiency improving composition with the various vesicle components and the Alcaligenes-produced polysaccharide used in Test 4. The application results were indicated in Table 7. In addition, a temperature cycle in which the temperature was increased to about 40° C. when using the ultrasonic diagnosis, and then returned to room temperature after use was repeated, and by simulating the cycle, 5 cycles of 40° C.×12 hours and 10° C.×12 hours for 5 days of temperature history was performed. The results were indicated in Table 8. As a control, the results of 10° C. constant×5 days were also indicated in Table 8.

TABLE 6

| No. | 14 | 15 | 16 | 17 | 18 | 19 | 20 | 21 | 22 |
|---|---|---|---|---|---|---|---|---|---|
| A-3 | 0.05 | 0.025 | 0.025 | 0.025 | 0.025 | 0.025 | 0.025 | 0.025 | 0 |
| A-6 | 0.05 | 0.025 | 0.025 | 0.025 | 0.025 | 0.025 | 0.025 | 0.025 | 0 |
| B-1 | 0 | 0.05 | 0 | 0 | 0 | 0 | 0 | 0 | 0 |
| B-2 | 0 | 0 | 0.05 | 0 | 0 | 0 | 0 | 0 | 0 |
| B-3 | 0 | 0 | 0 | 0.05 | 0 | 0 | 0 | 0 | 0 |
| B-4 | 0 | 0 | 0 | 0 | 0.05 | 0 | 0 | 0 | 0.1 |
| B-5 | 0 | 0 | 0 | 0 | 0 | 0.05 | 0 | 0 | 0 |
| B-6 | 0 | 0 | 0 | 0 | 0 | 0 | 0.05 | 0 | 0 |
| C-1 | 0 | 0 | 0 | 0 | 0 | 0 | 0 | 0.05 | 0 |
| G-1 | 99.9 | 99.9 | 99.9 | 99.9 | 99.9 | 99.9 | 99.9 | 99.9 | 99.9 |

TABLE 7

| | Examples | | | | | | | |
|---|---|---|---|---|---|---|---|---|
| | No. | | | | | | | |
| | 14 | 15 | 16 | 17 | 18 | 19 | 20 | 21 |
| Embodiments | First | First | First | First | First | First | First | First |
| | embodiment | embodiment | embodiment | embodiment | embodiment | embodiment | embodiment | embodiment |
| | (1) | (1) | (1) | (1) | (1) | (1) | (1) | (1) |
| Ultrasonic | Mixture | Mixture | Mixture | Mixture | Mixture | Mixture | Mixture | Mixture |
| transmission | 14 | 15 | 16 | 17 | 18 | 19 | 20 | 21 |
| efficiency | | | | | | | | |
| improving | | | | | | | | |
| composition | | | | | | | | |
| With or | Wipe | Wipe | Wipe | Wipe | Wipe | Wipe | Wipe | Wipe |
| without | off | off | off | off | off | off | off | off |
| wiping off | | | | | | | | |
| Ultrasonic | H-1 | H-1 | H-1 | H-1 | H-1 | H-1 | H-1 | H-1 |
| diagnostic gel | | | | | | | | |
| composition | | | | | | | | |
| Conductance | 458 | 486 | 474 | 465 | 501 | 488 | 485 | 470 |
| value (μS) | | | | | | | | |
| Vpp(mV) | 0.21 | 0.18 | 0.18 | 0.19 | 0.16 | 0.18 | 0.17 | 0.19 |

TABLE 8

| | | | | Comparative | | | | Comparative |
|---|---|---|---|---|---|---|---|---|
| | Examples | | | example | Examples | | | example |
| | No. | | | | | | | |
| | 22 | 23 | 24 | 4 | 25 | 26 | 27 | 5 |
| Embodiments | First | First | First | First | First | First | First | First |
| | embodiment | embodiment | embodiment | embodiment | embodiment | embodiment | embodiment | embodiment |
| | (1) | (1) | (1) | (1) | (1) | (1) | (1) | (1) |
| Ultrasonic | Mixture | Mixture | Mixture | Mixture | Mixture | Mixture | Mixture | Mixture |
| transmission | 14 | 18 | 21 | 22 | 14 | 18 | 21 | 22 |
| efficiency | | | | | | | | |
| improving | | | | | | | | |
| composition | | | | | | | | |
| Temperature | 10° C. | 10° C. | 10° C. | 10° C. | Cycle of | Cycle of | Cycle of | Cycle of |
| history | constant | constant | constant | constant | 40° C. × | 40° C. × | 40° C. × | 40° C. × |
| | for 5 | for 5 | for 5 | for 5 | 12 hours | 12 hours | 12 hours | 12 hours |
| | days | days | days | days | 10° C. × | 10° C. × | 10° C. × | 10° C. × |
| | | | | | 12 hours | 12 hours | 12 hours | 12 hours |
| | | | | | for 5 | for 5 | for 5 | for 5 |
| | | | | | days | days | days | days |
| With or | Wipe off | Wipe off | Wipe off | Wipe off | Wipe off | Wipe off | Wipe off | Wipe off |
| without | | | | | | | | |
| wiping off | | | | | | | | |
| Ultrasonic | H-1 | H-1 | H-1 | H-1 | H-1 | H-1 | H-1 | H-1 |
| diagnostic gel | | | | | | | | |
| composition | | | | | | | | |
| Conductance | 457 | 497 | 470 | 442 | 456 | 489 | 470 | 238 |
| value (μS) | | | | | | | | |
| Vpp(mV) | 0.21 | 0.16 | 0.19 | 0.21 | 0.21 | 0.17 | 0.19 | 0.28 |

From the results of Table 7, the conductance values and the ultrasonic reflection signal intensity of Examples 15 to 15 in which a vesicle forming component was further mixed and Example 21 in which the Alcaligenes-produced polysaccharide was mixed were excellent as compared to Example 14 of Mixture 14 in which only glycogen and dextrin were mixed, and a synergistic effect was observed when glycogen and dextrin were allowed to coexist with the vesicle forming component and the Alcaligenes-produced polysaccharide.

Further, from the results of Table 8, Comparative Example 4 of Mixture 22 in which a vesicle forming component was only mixed was not much different from the results of Mixtures 14, 18, and 21 in 10° C. constant×5 days of temperature history, but after 5 cycles of 40° C.×12 hours and 10° C.×12 hours for 5 days of temperature history, Mixture 22 was deteriorated in the conductance value and the intensity of the ultrasonic reflection signal intensity, as indicated in Comparative Example 5, as compared to Mixture 14 of glycogen and dextrin were mixed, Example 18 in which the vesicle forming component was further mixed, and Example 21 in which Alcaligenes-produced polysaccharide was mixed. This result suggests that the formed vesicle was not able to maintain the structure for the above temperature cycle. On the other hand, in Mixture 18 in which the vesicle forming component was further mixed with glycogen and dextrin, as indicated in Example 26, even after 5 cycles of 40° C.×12 hours and 10° C.×12 hours for 5 days of temperature history, the effect was stable, and a synergistic effect was also observed for the influence of the temperature history by coexisting glycogen, dextrin, and vesicle forming components.

(5) Test 5-Effect of Ultrasonic Diagnostic Gel Composition (Sensory Test)

Table 9 indicated the mixtures of the ultrasonic diagnostic gel composition in which the type and mixing amount of the gelling agent used in Test 5 were changed. This ultrasonic diagnostic gel composition was used by a technician in the same way as in actual ultrasonic diagnosis, so that the sensory evaluation was performed on 8 items: “slipperiness”, “excellence of stretch”, “inability to cling to the probe”, “jelly addition frequency”, “sag resistance”, “ease of wiping off”, “stickiness after wiping off”, and “moisturizing feeling after wiping off”. The application results were indicated in Table 10. The evaluation was expressed as 1: Normal, 2: Somewhat good, 3: Good.

TABLE 9

| No. | 23 | 24 | 25 | 26 | 27 | 28 | 29 | 30 | 31 | 32 | 33 | 34 |
|---|---|---|---|---|---|---|---|---|---|---|---|---|
| A-3 | 0.25 | 0.25 | 0.25 | 0.25 | 0.25 | 0.25 | 0.25 | 0.25 | 0.25 | 0.25 | 0.25 | 0.25 |
| B-4 | 0.25 | 0.25 | 0.25 | 0.25 | 0.25 | 0.25 | 0.25 | 0.25 | 0.25 | 0.25 | 0.25 | 0.25 |
| C-1 | 0.06 | 0.06 | 0.06 | 0.06 | 0.06 | 0.06 | 0.06 | 0.06 | 0.06 | 0.06 | 0.06 | 0.06 |
| D-1 | 0.05 | 0.1 | 0 | 0 | 0 | 0 | 0 | 0 | 0 | 0 | 0 | 0 |
| D-2 | 0 | 0 | 0.25 | 0.5 | 0 | 0 | 0 | 0 | 0 | 0 | 0 | 0 |
| D-3 | 0 | 0 | 0 | 0 | 2 | 5 | 0 | 0 | 0 | 0 | 0 | 0 |
| D-4 | 0 | 0 | 0 | 0 | 0 | 0 | 0.05 | 0.1 | 0.25 | 0.5 | 2 | 5 |
| E-1 | 5 | 5 | 5 | 5 | 5 | 5 | 5 | 5 | 5 | 5 | 5 | 5 |
| E-2 | 5 | 5 | 5 | 5 | 5 | 5 | 5 | 5 | 2 | 2 | 2 | 2 |
| E-4 | 10 | 10 | 10 | 10 | 10 | 10 | 10 | 10 | 3 | 3 | 3 | 3 |
| F-1 | 2 | 0 | 0 | 0 | 0 | 0 | 0 | 2 | 0 | 0 | 0 | 0 |
| F-2 | 0 | 0 | 0 | 0 | 0 | 0 | 2 | 0 | 0 | 0 | 0 | 0 |
| F-3 | 0 | 1 | 0 | 0 | 0 | 0 | 1 | 0 | 0 | 0 | 0 | 0 |
| G-1 | 77.39 | 78.34 | 79.19 | 78.94 | 77.44 | 77.44 | 76.39 | 77.34 | 89.19 | 88.94 | 87.44 | 84.44 |

TABLE 10

| | Examples | | | | | | | | | | | |
|---|---|---|---|---|---|---|---|---|---|---|---|---|
| | No. | | | | | | | | | | | |
| | 28 | 29 | 30 | 31 | 32 | 33 | 34 | 35 | 36 | 37 | 38 | 39 |
| Embodi- | Second | Second | Second | Second | Second | Second | Second | Second | Second | Second | Second | Second |
| ments | embodi- | embodi- | embodi- | embodi- | embodi- | embodi- | embodi- | embodi- | embodi- | embodi- | embodi- | embodi- |
| | ment | ment | ment | ment | ment | ment | ment | ment | ment | ment | ment | ment |
| Ultrasonic | Mixture | Mixture | Mixture | Mixture | Mixture | Mixture | Mixture | Mixture | Mixture | Mixture | Mixture | Mixture |
| transmission | 23 | 24 | 25 | 26 | 27 | 28 | 29 | 30 | 31 | 32 | 33 | 34 |
| efficiency | | | | | | | | | | | | |
| improving | | | | | | | | | | | | |
| composition | | | | | | | | | | | | |
| Slipperiness | 2 | 2 | 2 | 2 | 1 | 1 | 2 | 2 | 2 | 2 | 1 | 1 |
| Excellenceof | 1 | 2 | 3 | 2 | 2 | 1 | 1 | 2 | 3 | 2 | 2 | 1 |
| stretch | | | | | | | | | | | | |
| Inability | 2 | 2 | 2 | 2 | 1 | 1 | 2 | 2 | 2 | 2 | 1 | 1 |
| to cling to | | | | | | | | | | | | |
| the probe | | | | | | | | | | | | |
| Jelly | 1 | 2 | 3 | 3 | 2 | 2 | 1 | 2 | 3 | 3 | 2 | 2 |
| addition | | | | | | | | | | | | |
| frequency | | | | | | | | | | | | |
| Sag | 1 | 2 | 3 | 3 | 3 | 3 | 1 | 2 | 3 | 3 | 3 | 3 |
| resistance | | | | | | | | | | | | |
| Easeof | 2 | 2 | 2 | 2 | 1 | 1 | 2 | 2 | 2 | 2 | 1 | 1 |
| wiping off | | | | | | | | | | | | |
| Stickiness | 2 | 2 | 3 | 3 | 2 | 1 | 2 | 2 | 3 | 3 | 2 | 1 |
| after | | | | | | | | | | | | |
| wiping off | | | | | | | | | | | | |
| Moisturizing | 2 | 2 | 2 | 2 | 2 | 2 | 2 | 2 | 2 | 2 | 2 | 2 |
| feeling | | | | | | | | | | | | |
| after | | | | | | | | | | | | |
| wiping off | | | | | | | | | | | | |

From the results of Test 5, it was indicated that the ultrasonic diagnostic gel composition of the present invention was evaluated as “normal” or higher in workability at the time of ultrasonic diagnosis even if the type of gelling agent and the amount of the gelling agent were changed.

(6) Test 6-Determination by Ultrasonic Image

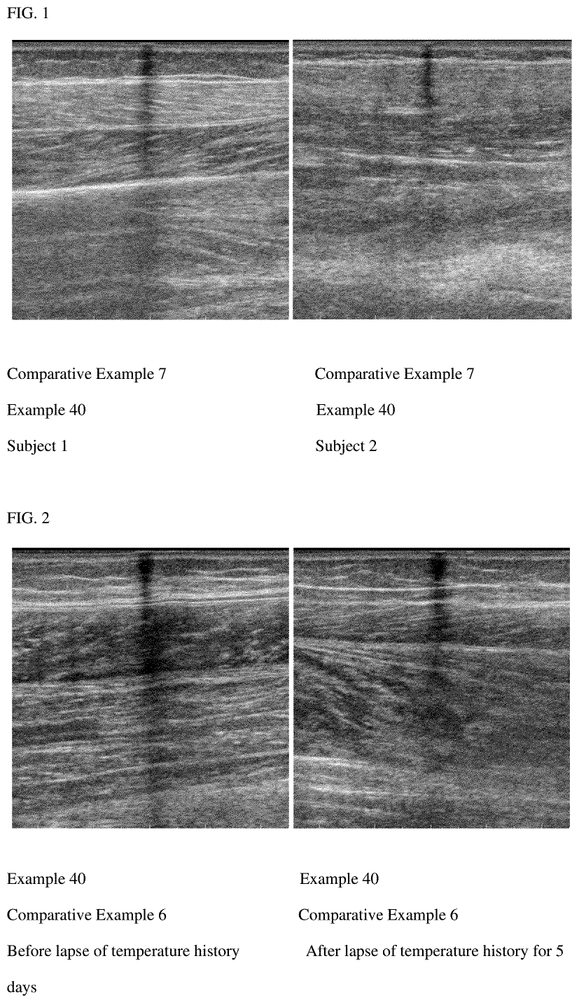

Table 11 indicated the mixtures (unit is weight (g)) corresponding to 300 g of the ultrasonic diagnostic gel composition used in Test 6. In addition, the conventional application-type contact medium H-1 was used. A determination method using ultrasonic images is a method performed in such a manner that the skin of the subject is divided into two parts with tape, the gel compositions to be compared are applied on either side, and then ultrasonic probes are applied to portions to be applied on both sides of the tape to capture the ultrasonic image of each portion, thereby comparing the captured images. The test subjects were two women, and the test sites were the abdomen and lower leg. The captured images were illustrated in FIGS. 1 to 5. Examples of applying the ultrasonic diagnostic gel composition of Mixtures Nos. 35 and 36 were set as Examples 40 and 41, respectively, and an example of applying the ultrasonic diagnostic gel composition of Mixture 37 was set as Comparative Example 6 and an example of applying the conventional application-type contact medium H-1 was set as Comparative Example 7.

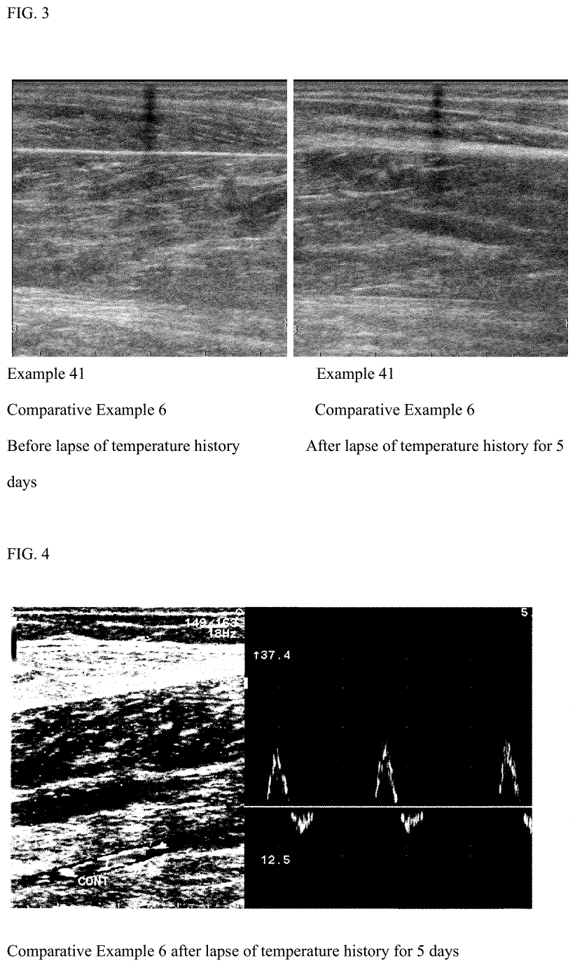

In FIGS. 1 to 3, black vertical lines in the center of the captured image indicate the position of the tape. In FIG. 1, the right side of the tape is the image of the example, and the left side is the image of the comparative example. In FIGS. 2 and 3, the left side of the tape is the image of the example, and the right side is the image of the comparative example.

TABLE 11

| Component types | No. | 35 | 36 | 37 |
|---|---|---|---|---|
| Hydrophilic fine particle forming | A-3 | 0.3 | 0.3 | 0 |
| component of the present | A-6 | 0 | 0.3 | 0 |
| invention | | | | |
| Vesicle forming component | B-1 | 0.3 | 0 | 0.3 |
| | B-4 | 0.8 | 0 | 1.1 |
| Alcaligenes-produced | C-1 | 0.15 | 0.15 | 0.15 |
| polysaccharide | | | | |
| Gelling agent | D-2 | 0 | 0.9 | 0 |
| | D-3 | 0.9 | 0 | 0.9 |
| | D-4 | 0 | 0.3 | 0 |
| Polyhydric alcohol | E-1 | 45 | 45 | 45 |
| | E-3 | 9 | 9 | 9 |
| | E-4 | 30 | 30 | 30 |
| Thickener | F-1 | 0.6 | 0.6 | 0.6 |
| Other components | J-1 | 0.6 | 0.6 | 0.6 |
| | J-2 | 0 | 0 | 0.6 |
| Purified water | G-1 | 208.15 | 207.25 | 207.55 |
| Neutralizing alkali | 10% | 4.2 | 5.6 | 4.2 |
| | KOH | | | |

FIG. 1 illustrates cross-sectional images (B images) of Example 40 and Comparative Example 7 for two subjects. By applying the ultrasonic diagnostic gel composition of the present invention, it was confirmed that the ultrasonic waves reached the lower part of the skin tissue in both subjects as compared with the case of applying the conventional application-type contact medium.

FIG. 2 illustrates cross-sectional images (B images) before and after the lapse of the temperature history of Example 40 and Comparative Example 6. Before the lapse of the temperature history of 5 cycles of 40° C.×12 hours and 10° C.×12 hours for 5 days, there was no difference in the captured images of Example 40 and Comparative Example 6, but after the lapse of the temperature history for 5 days, in Comparative Example 6 in which a vesicle forming component was only mixed, ultrasonic waves did not reach a deep portion of the skin; whereas in Example 40 in which the hydrophilic fine particle forming component and the vesicle forming component of the present invention were mixed, ultrasonic waves were able to reach a portion of the skin approximately the same as before the lapse of the temperature history, thereby indicating the coexistence effect of the hydrophilic fine particle forming component and the vesicle forming component.

FIG. 3 illustrates cross-sectional images (B images) before and after the lapse of the temperature history of Example 41 and Comparative Example 6. Before and after the lapse of the temperature history of 5 cycles of 40° C.×12 hours and 10° C.×12 hours for 5 days, there was almost no difference in the captured image of Example 41, but the captured image after the lapse of the temperature history of Comparative Example 6 indicated that the ultrasonic wave did not reach the deep portion of the skin, and the effect of the hydrophilic fine particles of the present invention was not influenced by the temperature history elapse, but the effect of the vesicle was influenced and decreased.

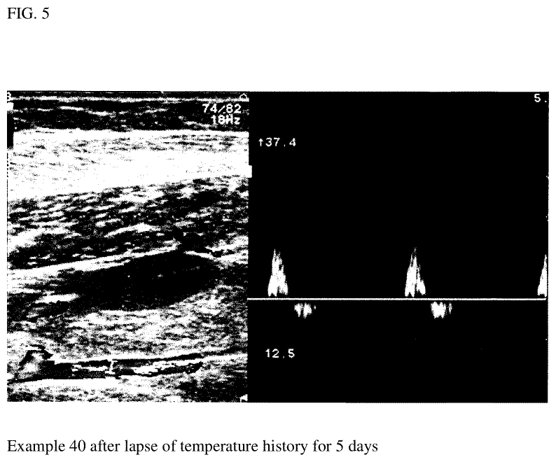

Furthermore, in order to evaluate the influence of the temperature history for 5 days on Example 40 and Comparative Example 6 in more detail, a comparison was made using Doppler display (color Doppler display, pulse Doppler display) images. FIG. 4 is an image of Comparative Example 6 after the lapse of the temperature history for 5 days, and FIG. 5 is an image of Example 40 after the lapse of the temperature history for 5 days. When comparing FIG. 4 with FIG. 5, as compared to the image in FIG. 4, the image in FIG. 5 is sharp, which can be confirmed up to the deep portion, and has a few multiple reflections and high Doppler signal intensity, and therefore, the ultrasonic diagnostic gel composition can provide a high-quality ultrasonic diagnostic image even through a temperature cycle during ultrasonic diagnosis.

1. An ultrasonic transmission efficiency improving composition comprising an aqueous solution containing hydrophilic fine particles including glycogen and/or hydrophilic fine particles including dextrin.

2. The ultrasonic transmission efficiency improving composition according to claim 1, wherein the glycogen is one or more selected from the group consisting of mussel glycogen, phytoglycogen, and enzyme-synthesized glycogen.

3. The ultrasonic transmission efficiency improving composition according to claim 1, wherein the dextrin is cyclic dextrin.

4. The ultrasonic transmission efficiency improving composition according to claim 1, wherein the hydrophilic fine particles are contained in an amount of 0.005% by weight to 3% by weight with respect to a total amount of the aqueous solution.

5. The ultrasonic transmission efficiency improving composition according to claim 1, wherein a particle size of the hydrophilic fine particle is in a range of 0.01 μm to 2.0 μm.

6. The ultrasonic transmission efficiency improving composition according to claim 1, wherein the aqueous solution contains an Alcaligenes-produced polysaccharide.

7. The ultrasonic transmission efficiency improving composition according to claim 1, wherein the aqueous solution contains a vesicle forming component that is a compound that forms a vesicle in water.

8. An ultrasonic diagnostic gel composition comprising:
the ultrasonic transmission efficiency improving composition according to claim 1; and
a carboxyvinyl polymer and/or a (meth)acrylic acid/(meth)acrylic acid ester copolymer and a salt thereof in a gel state.

9. The ultrasonic diagnostic gel composition according to claim 8, wherein a mixing amount of the carboxyvinyl polymer is 0.05% by weight to 5.0% by weight with respect to a total amount of the ultrasonic diagnostic gel composition, and a mixing amount of the (meth)acrylic acid/(meth)acrylic acid ester copolymer and the salt thereof is 0.05% by weight to 5.0% by weight with respect to the total amount of the ultrasonic diagnostic gel composition.

10. An ultrasonic imaging method comprising:
applying the ultrasonic transmission efficiency improving composition according to claim 1 to a skin; and
thereafter, applying an ultrasonic diagnostic gel composition that does not contain the ultrasonic transmission efficiency improving composition as it is to perform ultrasonic imaging, or
thereafter, wiping off the ultrasonic transmission efficiency improving composition and applying the ultrasonic diagnostic gel composition that does not contain the ultrasonic transmission efficiency improving composition to perform ultrasonic imaging.

11. An ultrasonic imaging method, wherein the ultrasonic diagnostic gel composition according to claim 8 is applied to the skin, and then ultrasonic imaging is performed as it is.
